(19) United States
(12) Patent Application Publication
(10) Pub. No.: US 2006/0104422 A1
(43) Pub. Date: May 18, 2006

(54) Transportation apparatus and tomography system

(75) Inventors: Shinichi Iisaku, Tokyo (JP); Shigeru Chikamatsu, Tokyo (JP); Kenichi Nishizawa, Tokyo (JP); Naoki Nakamura, Tokyo (JP); Akira Izuhara, Tokyo (JP); Katsumi Azu, Tokyo (JP); Masashi Maida, Tokyo (JP); Junko Sekiguchi, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/280,583

(22) Filed: Nov. 16, 2005

(30) Foreign Application Priority Data
Nov. 18, 2004 (JP) 2004-333826

(51) Int. Cl. A61B 6/04

(52) U.S. Cl. 378/209

(57) ABSTRACT

An object of the present invention is to concurrently perform the horizontal movements of a cradle and a table in a desirable manner and to desirably prevent interference of the table with a gantry. A microprocessor controls and interlocks a horizontal cradle movement mechanism and a horizontal table movement mechanism. The microprocessor concurrently performs the horizontal movements of the cradle and table. If an operator tries to vary a table height or a tilt angle, the microprocessor performs arithmetic operations to check if the table will interfere with the gantry at a current scan-time position, at which the table should be located during scanning, in the varied state. If the interference is predicted, the table is pulled out of the bore of the gantry until it reaches a position at which the table will not interfere with the gantry.

### CROSS REFERENCE TO RELATED APPLICATIONS

This application claims the benefit of Japanese Application No. 2004-333826 filed Nov. 18, 2004.

### BACKGROUND OF THE INVENTION

The present invention relates to transportation apparatus and a tomography system. More particularly, the present invention is concerned with transportation apparatus and a tomography system that can concurrently perform horizontal movement of a cradle and horizontal movement of a table in a desirable manner, and a tomography system that can desirably prevent interference with a gantry.

Transportation apparatus and an X-ray CT system that comprise a cradle which can be horizontally moved with a subject lying down thereon, a table which supports the cradle and can be horizontally moved, and a support which raises or lowers the table have been known in the past (refer to, for example, Patent Document 1).

[Patent Document 1] Japanese Unexamined Patent Application Publication No. 2004-208953

As for the foregoing conventional transportation apparatus and X-ray CT system, consideration has not been fully taken into the capability to concurrently perform horizontal movement of a cradle and horizontal movement of a table or the capability to prevent interference with a gantry.

### SUMMARY OF THE INVENTION

Therefore, an object of the present invention is to provide transportation apparatus and a tomography system that can concurrently perform horizontal movement of a cradle and horizontal movement of a table in a desirable manner, and transportation apparatus and a tomography system that can desirably prevent interference with a gantry.

According to the first aspect of the present invention, there is provided transportation apparatus comprising: a cradle which is horizontally moved with a subject lying down thereon; a table that supports the cradle so that the cradle can be horizontally moved; a support which bears the table so that the table can be horizontally moved and whose height can be varied; a horizontal cradle movement means for horizontally moving the cradle; a horizontal table movement means for horizontally moving the table; a table height variation means for varying the height of the table; and a control means for controlling and interlocking the horizontal cradle movement means and horizontal table movement means so that the horizontal movements of the cradle and table can be performed concurrently.

In the transportation apparatus according to the first aspect, the horizontal cradle movement means and horizontal table movement means are controlled and interlocked. Consequently, the horizontal movements of the cradle and table can be concurrently performed in a desirable manner.

According to the second aspect of the present invention, there is provided transportation apparatus having the same components as those of the transportation apparatus in accordance with the first aspect, wherein: the control means determines a target cradle position and a target table position; the horizontal table movement means horizontally moves the table until the table reaches the target table position; the horizontal cradle movement means horizontally moves the cradle until the cradle reaches the target cradle position; and the velocities of the horizontal movements are controlled so that the arrivals of the table and cradle will coincide with each other.

In the transportation apparatus according to the second aspect, the horizontal movements of the cradle and table are simultaneously stopped. An operator accurately recognizes the stoppages of the cradle and table. This leads to improved safety.

According to the third aspect of the present invention, there is provided transportation apparatus having the same components as those of the transportation apparatus in accordance with the first aspect, wherein when decelerating the table, the control means accelerates the cradle so as to compensate for the deceleration.

In the transportation apparatus according to the third aspect, even when the table is decelerated, a velocity of horizontal movement a subject perceives remains constant. The subject will therefore not undergo discomfort derived from a variation of the velocity.

According to the fourth aspect of the present invention, there is provided transportation apparatus having the same components as those of the transportation apparatus in accordance with the first aspect, wherein when horizontally moving the table, the control means concurrently horizontally moves the cradle in an opposite direction by the magnitude of the horizontal movement made by the table.

In the transportation apparatus according to the fourth aspect, the table can be moved horizontally with the cradle held at a certain position.

According to the fifth aspect of the present invention, there is provided a tomography system comprising: a gantry that produces tomographic images of a subject; a cradle which carries the subject, who lies down thereon, into the bore of the gantry; a table which supports the cradle so that the cradle can be horizontally moved; a support which bears the table so that the table can be horizontally moved and whose height can be varied; a horizontal cradle movement means for horizontally moving the cradle; a horizontal table movement means for horizontally moving the table; a table height variation means for varying the height of the table; and a control means for controlling and interlocking the horizontal cradle movement means and horizontal table movement means so that the horizontal movements of the cradle and table can be performed concurrently.

In the tomography system according to the fifth aspect, the horizontal cradle movement means and horizontal table movement means are controlled and interlocked. Consequently, the horizontal movements of the cradle and table can be performed concurrently in a desirable manner.

According to the sixth aspect of the present invention, there is provided a tomography system having the same components as those of the tomography system in accordance with the fifth aspect, wherein: the control means determines a target cradle position and a target table position; the horizontal table movement means horizontally moves the table until the table reaches the target table position; the horizontal cradle movement means horizontally moves the cradle until the cradle reaches the target cradle position; and the velocities of the horizontal movements are controlled so that the arrivals of the table and cradle will coincide with each other.

In the tomography system according to the sixth aspect, the horizontal movements of the cradle and table are stopped simultaneously. Consequently, an operator can accurately recognize the stoppages of the cradle and table. This leads to improved safety.

According to the seventh aspect of the present invention, there is provided a tomography system having the same components as those of the tomography system in accordance with the fifth aspect, wherein when decelerating the table, the control means accelerates the cradle so as to compensate for the deceleration.

In the tomography system according to the seventh aspect, even when the table is decelerated, the velocity of horizontal movement a subject perceives remains constant. Consequently, the subject will not undergo discomfort derived from a variation of the velocity.

According to the eighth aspect of the present invention, there is provided a tomography system having the same components as those of the tomography system in accordance with the fifth aspect, wherein: when horizontally moving the table, the control means concurrently horizontally moves the cradle in an opposite direction by the magnitude of the horizontal movement made by the table.

In the tomography system according to the eight aspect, the table can be moved horizontally with the cradle held at a certain position.

According to the ninth aspect of the present invention, there is provided a tomography system comprising: a gantry that produces tomographic images of a subject; a cradle which carries the subject, who lies down thereon, into the bore of the gantry; a table which supports the cradle so that the cradle can be horizontally moved; a support which bears the table so that the table can be horizontally moved and whose height can be varied; a horizontal cradle movement means for horizontally moving the cradle; a horizontal table movement means for horizontally moving the table; a table height variation means for varying the height of the table; and a control means for controlling the horizontal cradle movement means and horizontal table movement means. The control means detects a position, at which the table lies closest to the gantry but does not interfere with the gantry, on the basis of a current tilt angle and a current table height, and regards the position as a scan-time position at which the table should be located during scanning. When the tilt angle or table height is varied, if the interference of the table with the gantry is predicted, the table is pulled out of the bore of the gantry.

In the tomographic system according to the ninth aspect, when a current state is sustained, an optimal scan-time position can be determined. If a tilt angle or a table height is varied, interference of the table with the gantry can be prevented desirably.

According to the tenth aspect of the present invention, there is provided a tomography system comprising: a gantry that produces tomographic images of a subject; a cradle which carries the subject, who lies down thereon, into the bore of the gantry; a table which supports the cradle so that the cradle can be horizontally moved; a support which bears the table so that the table can be horizontally moved and whose height can be varied; a horizontal cradle movement means for horizontally moving the cradle; a horizontal table movement means for horizontally moving the table; a table height variation means for varying the height of the table; and a control means for controlling the horizontal cradle movement means and horizontal table movement means. The control means determines a scan-time position, at which the table should be located during scanning, on the basis of a region to be scanned or the conditions for scanning. When a tilt angle or a table height is varied, if the scan-time position at which the table should be located during scanning is predicted to become improper, the table is pulled out of the bore of the gantry.

In the tomography system according to the tenth aspect, as long as a planned region to be scanned or conditions for scanning such as a protocol for scanning are sustained, an optimal scan-time position can be determined. Even if a tilt angle or a table height is varied, interference of the table with the gantry can be prevented desirably.

According to the eleventh aspect of the present invention, there is provided a tomography system comprising: a gantry that produces tomographic images of a subject; a cradle which carries the subject, who lies down thereon, into the bore of the gantry; a table which supports the cradle so that the cradle can be horizontally moved; a support which bears the table so that the table can be horizontally moved and whose height can be varied; a horizontal cradle movement means for horizontally moving the cradle; a horizontal table movement means for horizontally moving the table; a table height variation means for varying the height of the table; and a control means for controlling the horizontal cradle movement means and horizontal table movement means. The control means detects a position, at which the table will not interfere with the gantry but will lie closest to the gantry, on the basis of a maximum tilt angle and a current table height, and regards the position as a scan-time position at which the table should be located during scanning. Moreover, when the table height is varied, if the interference of the table with the gantry is predicted, the table is pulled out of the bore of the gantry.

In the tomography system according to the eleventh aspect, a scan-time position at which the table should be located during scanning and at which even if the gantry is tilted at the maximum tilt angle, the table will not interfere with the gantry can be determined. Even if the table height is varied, the interference of the table with the gantry can be prevented desirably.

According to the twelfth aspect of the present invention, there is provided a tomography system comprising: a gantry that produces tomographic images of a subject; a cradle which carries the subject, who lies down thereon, into the bore of the gantry; a table which supports the cradle so that the cradle can be horizontally moved; a support which bears the table so that the table can be horizontally moved and whose height can be varied; a horizontal cradle movement means for horizontally moving the cradle; a horizontal table movement means for horizontally moving the table; a table height variation means for varying the height of the table; and a control means for controlling the horizontal cradle movement means and horizontal table movement means. The control means detects a position, at which the table will not interfere with the gantry but will lie closest to the gantry, on the basis of a current tilt angle and a minimum table height permitting the cradle to be put into the bore at present, and regards it as a scan-time position at which the table should be located during scanning. When a tilt angle is varied, if the interference of the table with the gantry is predicted, the table is pulled out of the bore of the gantry.

In the tomography system according to the twelfth aspect, a scan-time position at which the table should be located during scanning and at which even if the table is lowered to the minimum table height permitting the cradle to be put into the bore of the gantry at present, the table will not interfere with the gantry can be determined. Even if the tilt angle is varied, the interference of the table with the gantry can be prevented desirably.

According to the thirteenth aspect of the present invention, there is provided a tomography system comprising: a gantry that produces tomographic images of a subject; a cradle which carries the subject, who lies down thereon, into the bore of the gantry; a table which supports the cradle so that the cradle can be horizontally moved; a support which bears the table so that the table can be horizontally moved and whose height can be varied; a horizontal cradle movement means for horizontally moving the cradle; a horizontal table movement means for horizontally moving the table; a table height variation means for varying the height of the table; and a control means for controlling the horizontal cradle movement means and horizontal table movement means. The control means detects a position, at which the table will not interfere with the gantry but will lie closest to the gantry, on the basis of a maximum tilt angle and a minimum table height permitting the cradle to be put into the bore of the gantry with the gantry held at the maximum tilt angle, and regards it as a scan-time position at which the table should be located during scanning.

In the tomography system according to the thirteenth aspect, the position at which the table will not interfere with the gantry even under the worst conditions is determined as the scan-time position at which the table should be located during scanning. The interference of the table with the gantry can be prevented at any tilt angle or at any table height (which is equal to or larger than a minimum table height permitting the cradle to be put into the bore of the gantry with the gantry held at the maximum tilt angle). If a scanning permissible range must be enlarged, the table may be put into the bore of the gantry according to a region to be scanned, an actual tilt angle, or an actual table height to such an extent that the table will not interfere with the gantry.

According to the fourteenth aspect of the present invention, there is provided a tomography system having the same components as those of the tomography system in accordance with any of the ninth to thirteenth aspects, wherein when a tilt angle or a table height is varied, if interference of the cradle with the gantry is predicted, the control means suspends the variation of the tilt angle or table height.

In the tomography system according to the fourteenth aspect, if interference of the cradle with the gantry is predicted, the variation of the tilt angle or table height is suspended. The interference of the cradle with the gantry can therefore be prevented.

According to the fifteenth aspect of the present invention, there is provided a tomography system having the same components as those of the tomography system in accordance with any of the fifth to fourteenth aspects, wherein the table has two or more table portions.

The table may be of a nested telescopic type. In this case, the fifth to fourteenth aspects are adapted to a table portion that is put into the innermost part of the bore of the gantry.

According to the sixteenth aspect of the present invention, there is provided a tomography system having the same components as those of the tomography system in accordance with any of the fifth to fifteenth aspects, wherein the tomography system is an X-ray CT system.

In the tomography system according to the sixteenth aspect, the horizontal movements of the cradle and table can be performed concurrently in a desirable manner for the purpose of X-ray CT. Moreover, interference of the table with the gantry can be prevented desirably.

According to the seventeenth aspect of the present invention, there is provided a tomography system having the same components as those of the tomography system in accordance with the sixteenth aspect, wherein the control means concurrently horizontally moves the table and cradle during scout scanning, axial cluster scanning, or helical scanning.

In the tomography system according to the seventeenth aspect, a scanning permissible range can be expanded for scout scanning, axial cluster scanning, or helical scanning.

What is referred to as axial cluster scanning is a technique of continuously performing axial scanning at different slicing positions.

According to the transportation apparatus and tomography system in which the present invention is implemented, the horizontal movements of the cradle and table can be performed concurrently in a desirable manner. Moreover, interference of the table with the gantry can be prevented desirably.

The transportation apparatus and tomography system in accordance with the present invention can be adapted to an X-ray CT system or an MR imaging system.

Further objects and advantages of the present invention will be apparent from the following description of the preferred embodiments of the invention as illustrated in the accompanying drawings.

### BRIEF DESCRIPTION OF THE DRAWINGS

FIG. 1 shows the configuration of an X-ray CT system in accordance with the first embodiment of the present invention.

FIG. 2 is an explanatory diagram concerning the first case where the horizontal movements of a cradle and a table are controlled and interlocked according to the present invention.

FIG. 3 is an explanatory diagram concerning the second case where the horizontal movements of the cradle and table are controlled and interlocked according to the present invention.

FIG. 4 is the first explanatory diagram concerning the first case where interference of the table with a gantry is prevented according to the present invention.

FIG. 5 is the second explanatory diagram concerning the first case where the interference of the table with the gantry is prevented according to the present invention.

FIG. 6 is the third explanatory diagram concerning the first case where the interference of the table with the gantry is prevented according to the present invention.

FIG. 7 is an explanatory diagram concerning the third case where the horizontal movements of the cradle and table are controlled and interlocked according to the present invention.

FIG. 8 is the first explanatory diagram concerning the second case where the interference of the table with the gantry is prevented according to the present invention.

FIG. 9 is the second explanatory diagram concerning the second case where the interference of the table with the gantry is prevented according to the present invention.

FIG. 10 is the third explanatory diagram concerning the second case where the interference of the table with the gantry is prevented according to the present invention.

FIG. 11 is the first explanatory diagram concerning the third case where the interference of the table with the gantry is prevented according to the present invention.

FIG. 12 is the second explanatory diagram concerning the third case where the interference of the table with the gantry is prevented according to the present invention.

FIG. 13 is the first explanatory diagram concerning the fourth case where the interference of the table with the gantry is prevented according to the present invention.

FIG. 14 is the second explanatory diagram concerning the fourth case where the interference of the table with the gantry is prevented according to the present invention.

FIG. 15 is an explanatory diagram concerning the fifth case where the interference of the table with the gantry is prevented according to the present invention.

### DETAILED DESCRIPTION OF THE INVENTION

The present invention will be detailed below by taking illustrated embodiments for instance. Noted is that the present invention will not be limited to the embodiments.

### FIRST EMBODIMENT

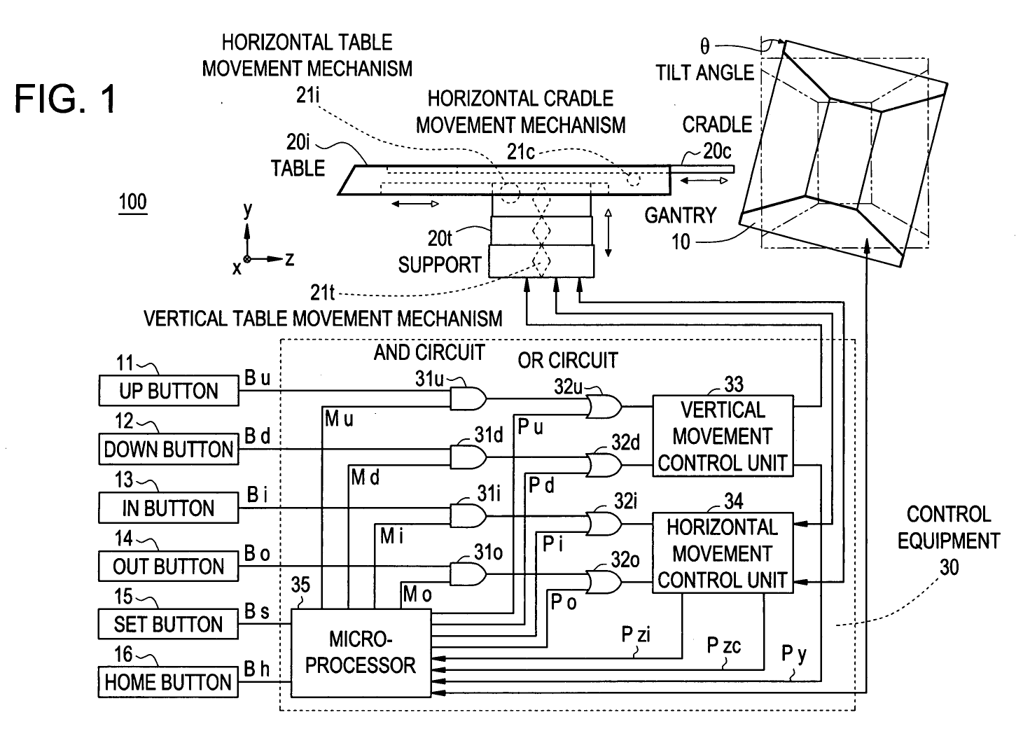

FIG. 1 is a perspective view showing the major portion of an X-ray CT system 100 in accordance with the first embodiment.

The X-ray CT system 100 comprises: a gantry 10 that produces tomographic images of a subject; a cradle 20c which carries the subject, who lies down thereon, into the bore of the gantry 10; a table 20i which supports the cradle 20c so that the cradle 20c can be horizontally moved; a support 20t which bears the table 20i so that the table 20i can be horizontally moved and whose height can be varied; a horizontal cradle movement mechanism 21c which horizontally moves the cradle 20c; a horizontal table movement mechanism 21i which horizontally moves the table 20i; a vertical table movement mechanism 21t which varies the height of the table 20i; control equipment 30 which controls the horizontal cradle movement mechanism 21c, horizontal table movement mechanism 21i, and vertical table movement mechanism 21t; and an Up button 11, a Down button 12, an In button 13, an Out button 14, a Set button 15, and a Home button 16 which an operator handles.

When the Up button 11 is released, a signal Bu goes low. When the Up button 11 is depressed, the signal Bu goes high. When the Down button 12 is released, a signal Bd goes low. When the Down button 12 is depressed, the signal Bd goes high. When the In button 13 is released, a signal Bi goes low. When the In button 13 is depressed, the signal Bi goes high. When the Out button 14 is released, a signal Bo goes low. When the Out button 14 is depressed, the signal Bo goes high.

When the Set button 15 is released (turned off), a signal Bs goes low. When the Set button 15 is depressed (turned on), the signal Bs goes high. The signal Bs is transferred to a microprocessor 35.

When the Home button 16 is released, a signal Bh goes low. When the Home button 16 is depressed, the signal Bh goes high. The signal Bh is transferred to the microprocessor 35.

The microprocessor 35 monitors a vertical table position Py fed back from a vertical movement control unit 33. In order to suspend raising of the table initiated with the Up button 11, the signal Mu is driven low. In order to permit the raising, the signal Mu is driven high. In order to suspend lowering of the table initiated with the Down button 12, the signal Md is driven low. In order to permit the lowering, the signal Md is driven high. Furthermore, the microprocessor 35 monitors a horizontal cradle position Pzc and a horizontal table position Pzi which are fed back from a horizontal movement control unit 34. In order to suspend putting of the cradle 20c and/or table 20i into the bore of the gantry initiated with the In button 13 (horizontal movement to be performed in a direction of putting the cradle and/or table into the bore of the gantry 10), the signal Mi is driven low. In order to permit the putting, the signal Mi is driven high. Moreover, in order to suspend pulling of the cradle 20c and/or table 20i out of the bore of the gantry initiated with the Out button 14 (horizontal movement to be performed in a direction of pulling the cradle and/or table out of the bore of the gantry 10), the signal Mo is driven low. In order to permit the pulling, the signal Mo is driven high.

Moreover, the microprocessor 35 drives the signals Pu, Pd, Pi, and Po to the high or low level so as to initiate or suspend setup of the cradle and/or table performed in response to the press of the Set button 15, returning of the cradle and/or table to the home position performed in response to the press of the Home button 16, or horizontal movement of the cradle 20c and/or table 20i performed for scout scanning, axial cluster scanning, or helical scanning.

An AND circuit 31u calculates an AND of the signals Bu and Mu. The AND and signal Pu are transferred to the vertical movement control unit 33 via an OR circuit 30u circuit 32u. An AND circuit 31d calculates an AND of the signals Bd and Md. The AND and signal Pd are transferred to the vertical movement control unit 33 via an OR circuit 30d circuit 32d. An AND circuit 31i calculates an AND of the signals Bi and Mi. The AND and signal Pi are transferred to the horizontal movement control unit 34 via an OR circuit 30i circuit 32i. An AND circuit 31o calculates an AND of the signals Bo and Mo. The AND and signal Po are transferred to the horizontal movement control unit 34 via an OR circuit 30o circuit 32o.

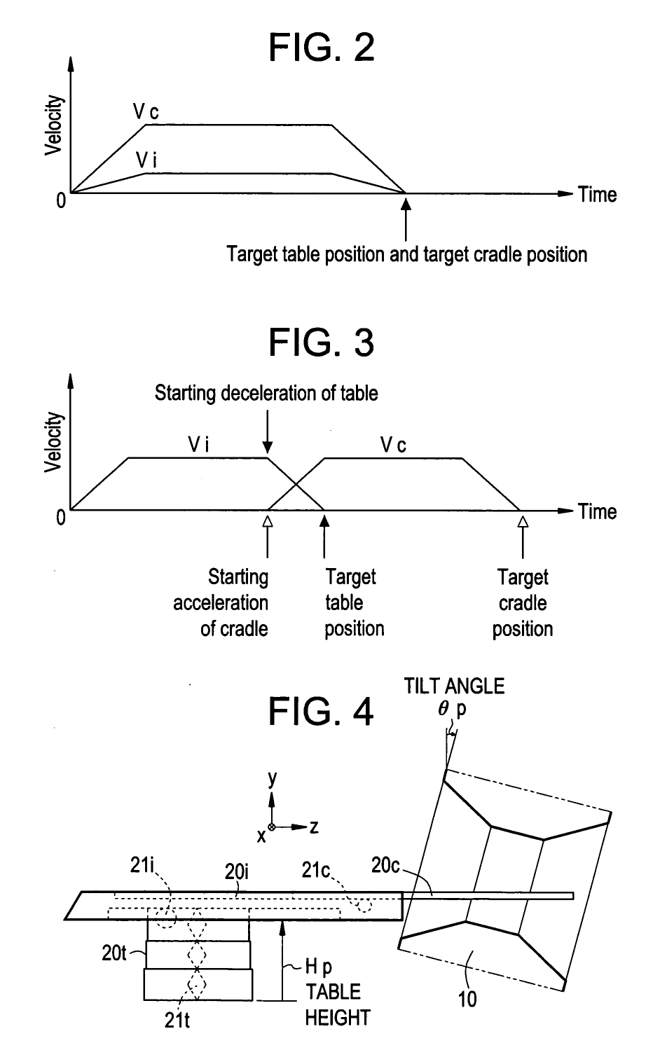

If an input from the OR circuit 30u circuit 32u is a high-level signal, the vertical movement control unit 33 controls the vertical table movement mechanism 21t so that the table 20i will be raised. If an input from the OR circuit 30d circuit 32d is a high-level signal, the vertical movement control unit 33 controls the vertical table movement mechanism 21t so that the table 20i will be lowered. If an input from the OR circuit 30i circuit 32i is a high-level signal, the horizontal movement control unit 34 controls the horizontal cradle movement mechanism 21c and/or horizontal table movement mechanism 20i so that the cradle 20c and/or table 20i will be put into the bore of the gantry. If an input from the OR circuit 30c circuit 32o is a high-level signal, the horizontal movement control unit 34 controls the horizontal cradle movement mechanism 21c and/or horizontal table movement mechanism 20i so that the cradle 20c and/or table 20i will be pulled out of the bore of the gantry. FIG. 2 is an explanatory diagram concerning the first case where the microprocessor 35 controls and interlocks the horizontal cradle movement mechanism 21c and horizontal table movement mechanism 21i.

When the Set button 15 or Home button 16 is depressed, the microprocessor 35 controls the horizontal cradle movement mechanism 21c so that the cradle 20c will be moved from a current cradle position toward a target cradle position until the cradle reaches the target cradle position. Concurrently, the microprocessor 35 controls the horizontal table movement mechanism 21i so that the table 20i will be moved from a current table position toward a target table position until the table reaches the target table position. The microprocessor 35 then controls a horizontal table movement velocity Vi and a cradle movement velocity Vc so that the arrival of the table 20i and the arrival of the cradle 20c will coincide with each other.

Incidentally, the target cradle position and target table position correspond to scan-time positions at which the cradle and table should be located during scanning after they are brought to setup states. After the cradle and table are brought to home states, the target cradle position and target table position are positions optimal for a subject to ride or get off the cradle 20c.

When the horizontal cradle movement mechanism and horizontal table movement mechanism are controlled and interlocked as mentioned above, the horizontal movements of the cradle 20c and table 20i are stopped simultaneously. An operator can accurately recognize the completion of horizontal movements. This leads to improved safety.

FIG. 3 is an explanatory diagram concerning the second case where the microprocessor 35 controls and interlocks the horizontal cradle movement mechanism 1c and horizontal table movement mechanism 21i.

When the Set button 15 or Home button 16 is depressed, the microprocessor 35 controls the horizontal table movement mechanism 21i so that the table 20i will be moved from a current table position toward a target table position until the table 20i reaches the target position. Concurrently, the microprocessor 35 controls the horizontal cradle movement mechanism 21c so that the cradle 20c will be accelerated in order to compensate for deceleration of the table 20i. Thus, the cradle 20c is moved from a current cradle position toward a target cradle position until the cradle reaches the target position.

Incidentally, the target cradle position and target table position correspond to scan-time positions at which the cradle and table should be located during scanning after they are brought to the setup states. When the cradle and table are brought to the home states, the target cradle position and target table position are positions optimal for a subject to ride or get off the cradle 20c.

When the horizontal cradle movement mechanism 21c and horizontal table movement mechanism 21i are controlled and interlocked as mentioned above, even if the table 20i is decelerated, a horizontal movement velocity a subject perceives remains constant. The subject will not undergo discomfort derived from a variation of the velocity.

FIG. 4 to FIG. 7 are explanatory diagrams concerning the first case where the microprocessor 35 prevents interference of the table 20i with the gantry 10.

As shown in FIG. 4, the microprocessor 35 monitors a current tilt angle Dp and a current table height Hp. The microprocessor 35 calculates a position, at which the table 20i will not interfere with the gantry 10 but will lie closest to the gantry 10, under the conditions of the tilt angle Dp and table height Hp, and regards the position as a scan-time position at which the table 20i should be located during scanning. For scanning, the table 20i is put into the bore of the gantry until it reaches the scan-time position.

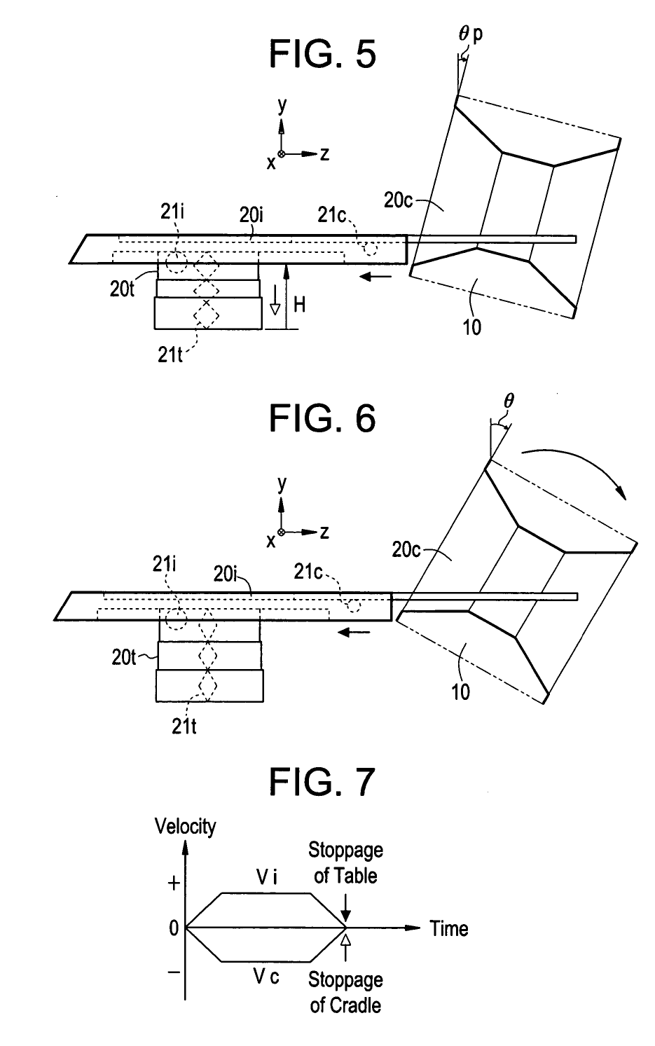

As shown in FIG. 5, when an operator tries to vary the table height H, the microprocessor 35 performs arithmetic operations to check if the table 20i will interfere with the gantry 10 at the varied table height H. If the interference is predicted, the table is pulled out of the bore of the gantry until it reaches a position at which the table will not interfere with the gantry. Thereafter, the microprocessor permits the variation of the table height.

As shown in FIG. 6, if an operator tries to vary a tilt angle θ, the microprocessor performs arithmetic operations to check if the table 20i will interfere with the gantry 10 with the gantry 10 at the varied tilt angle θ. If the interference is predicted, the table 20i is pulled out of the bore of the gantry until it reaches a position at which the table will not interfere with the gantry. Thereafter, the microprocessor permits the variation of the tilt angle.

When the table 20i is controlled as mentioned above, the interference of the table 20i with the gantry 10 can be prevented desirably.

As seen from FIG. 7, when the microprocessor 35 pulls the table 20i out of the bore of the gantry, the microprocessor 35 concurrently puts the cradle 20c into the bore by moving it in an opposite direction by the magnitude of horizontal movement made by the table 20i.

Consequently, while the cradle 20c is held at a certain position, the table 20i is pulled out of the bore of the gantry.

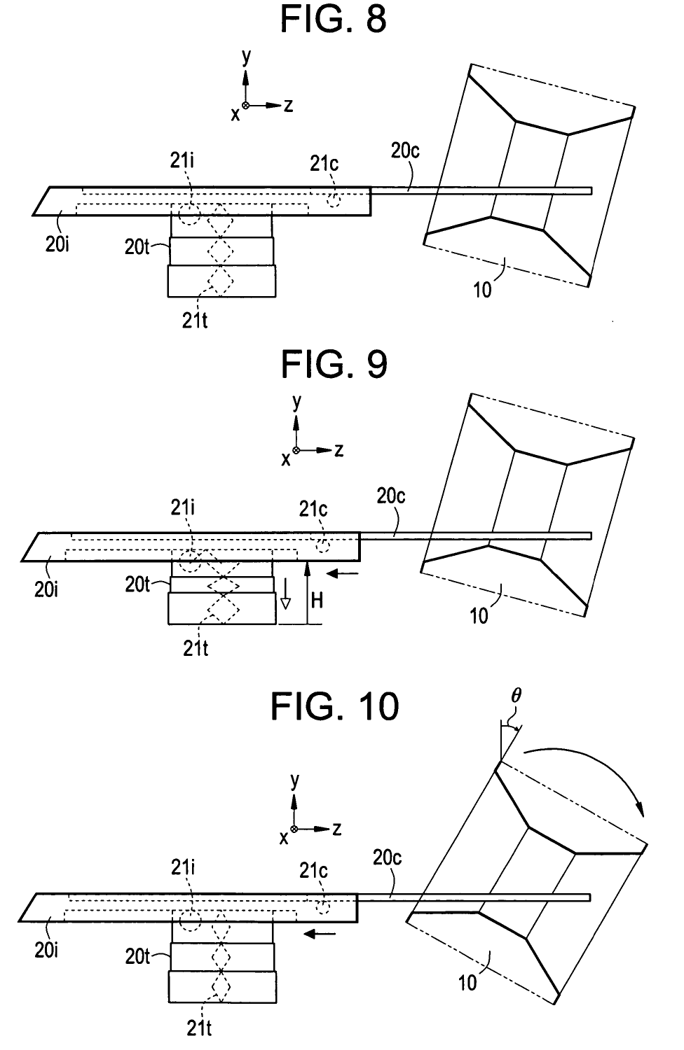

FIG. 8 to FIG. 10 are explanatory diagrams concerning the second case where the microprocessor 35 prevents interference of the table 20i with the gantry 10.

The microprocessor 35 determines a scan-time position, at which the table 20c should be located during scanning, on the basis of a region to be scanned or a protocol for scanning which is determined in course of planning a scan.

For example, when puncture is performed, a position separated by about 50 cm from the gantry 10 is, as shown in FIG. 8, regarded as a scan-time position. This is intended not to allow the table 20i to interfere in the work of puncture. When a scan is performed, the table 20i is put into the bore of the gantry until it reaches the scan-time position. As shown in FIG. 9, if an operator tries to vary a table height H, the microprocessor 35 performs arithmetic operations to check if the table 20i can be separated by the distance of about 50 cm from the gantry 10 at the varied table height H. If the microprocessor 35 predicts that the table will not be separated by the distance of about 50 cm from the gantry, the microprocessor 35 pulls the table 20i out of the bore of the gantry until the table reaches a position at which the table is separated by the distance of about 50 cm from the gantry. Thereafter, the microprocessor permits the variation of the table height. Moreover, as shown in FIG. 10, if an operator tries to vary a tilt angle θ, the microprocessor performs arithmetic operations to check if the table 20i will be separated by the distance of about 50 cm from the gantry 10 with the gantry set at the varied tilt angle θ. If the microprocessor predicts that the table will not be separated by the distance of about 50 cm from the gantry, the microprocessor pulls the table 20i out of the bore of the gantry until the table reaches a position at which the table will be separated by the distance of about 50 cm from the gantry. Thereafter, the microprocessor permits the variation of the tilt angle.

When the table 20i is controlled as mentioned above, the interference of the table 20i with the gantry 10 can be prevented desirably.

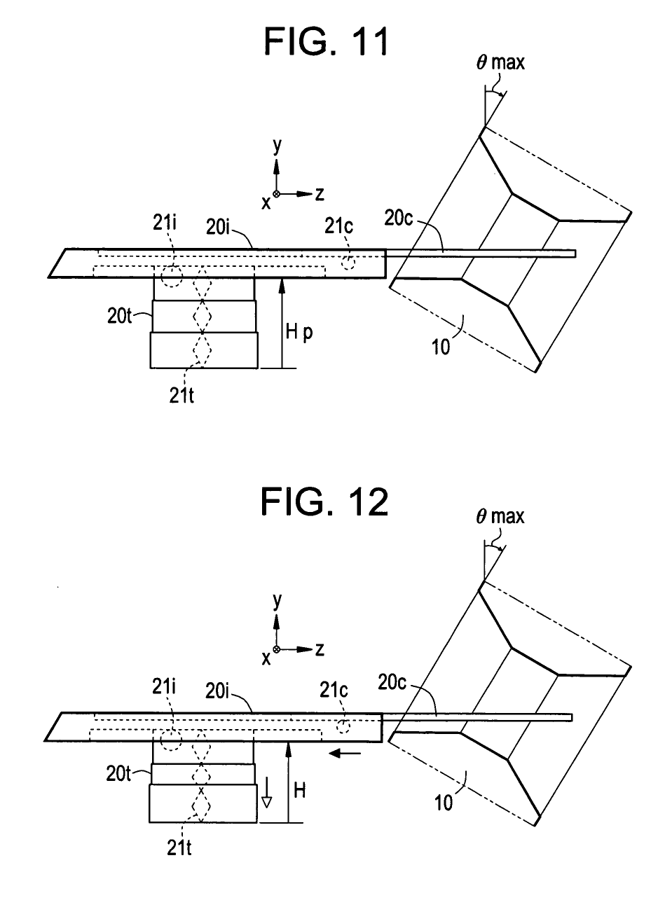

FIG. 11 and FIG. 12 are explanatory diagrams concerning the third case where the microprocessor 35 prevents interference of the table 20i with the gantry 10.

As shown in FIG. 11, the microprocessor 35 monitors a current table height Hp, and calculates a position, at which the table 20i will not interfere with the gantry 10 but will lie closest to the gantry 10, under the conditions of the table height Hp and a maximum tilt angle Dmax. The microprocessor 35 then regards the position as a scan-time position at which the table 20i should be located during scanning. For scanning, the microprocessor puts the table 20i into the bore of the gantry until the table reaches the scan-time position.

As shown in FIG. 12, if an operator tries to vary the table height H, the microprocessor performs arithmetic operations to check if the table 20i will interfere with the gantry at the varied table height H with the gantry set at the maximum tilt angle Dmax. If the interference is predicted, the table 20i is pulled out of the bore of the gantry until the table reaches a position at which the table will not interfere with the gantry. Thereafter, the microprocessor permits the variation of the table height.

Since the maximum tilt angle Dmax is adopted as one of conditions, even if a tilt angle is varied arbitrarily, the table 20i will not interfere with the gantry 10.

When the table 20i is controlled as mentioned above, interference of the table 20i with the gantry 10 can be prevented desirably.

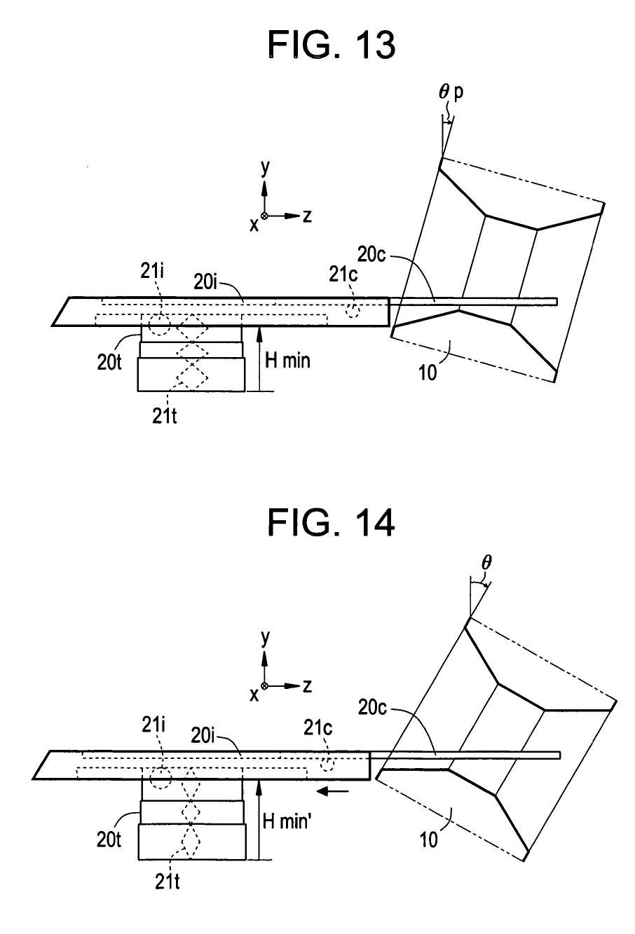

FIG. 13 and FIG. 14 are explanatory diagrams concerning the fourth case where the microprocessor 35 prevents interference of the table 20i with the gantry 10.

As shown in FIG. 13, the microprocessor 35 monitors a current tilt angle θp. The microprocessor calculates a position, at which the table 20i will not interfere with the gantry 10 but will lie closest to the gantry 10, under the conditions of the current tilt angle θp and a minimum table height Hmin permitting the cradle 20c to be put into the bore of the gantry with the gantry set at the tilt angle θp. The microprocessor regards the position as a scan-time position at which the table 20i should be located during scanning. For scanning, the table 20i is put into the bore of the gantry until it reaches the scan-time position.

As shown in FIG. 14, if an operator tries to vary a tilt angle θ, the microprocessor performs arithmetic operations to check if the table 20i will interfere with the gantry 10 under the conditions of the varied tilt angle θ and a minimum table height Hmin′ permitting the cradle 20c to be put into the bore of the gantry with the gantry set at the tilt angle θ. If the microprocessor predicts the interference, the microprocessor pulls the table 20i out of the bore of the gantry until the table reaches a position at which the table will not interfere with the gantry. Thereafter, the microprocessor permits the variation of the tilt angle.

Since the minimum table height Hmin or Hmin′ permitting the cradle 20c to be put into the bore of the gantry is adopted as one of conditions, even if the table height H is varied arbitrarily (needless to say, the table height is equal to or larger than the minimum table height Hmin or Hmin′), the table 20i will not interfere with the gantry 10.

When the table 20i is controlled as mentioned above, interference of the table 20i with the gantry 10 can be prevented desirably.

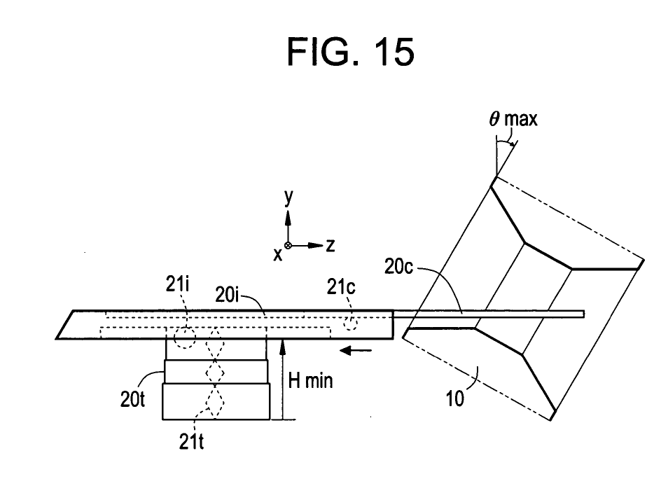

FIG. 15 is an explanatory diagram concerning the fifth case where the microprocessor 35 prevents interference of the table 20i with the gantry 10.

As shown in FIG. 15, the microprocessor 35 calculates a position, at which the table 20i will not interfere with the gantry 10 but will lie closest to the gantry 10, under the conditions of a maximum tilt angle θmax and a minimum table height Hmin permitting the cradle 20c to be put into the bore of the gantry with the gantry set at the maximum tilt angle θmax, and regards the position as a scan-time position at which the table 20i should be located during scanning. For scanning, the table 20i is put into the bore of the gantry until it reaches the scan-time position.

Since the maximum tilt angle θmax and the minimum table height Hmin permitting the cradle 20c to be put into the bore of the gantry with the gantry set at the maximum tilt angle θmax are adopted as conditions, even if a tilt angle θ or a table height H (that is, needless to say, equal to or larger than the minimum table height Hmin) is varied arbitrarily, the table 20i will not interfere with the gantry 10.

When the table 20i is controlled as mentioned above, interference of the table 20i with the gantry 10 can be prevented desirably.

If a scanning permissible range must be expanded, the table 20i may be put into the bore of the gantry according to a region to be scanned, an actual tilt angle, or an actual table height to such an extent that the table 20i will not interfere with the gantry 10.

If an operator tries to vary a table height or a tilt angle, the microprocessor 35 prevents interference of the table 20i with the gantry 10 as described previously. The microprocessor 35 performs arithmetic operations to check under the conditions of the varied table height or tilt angle, the position of the table 20i whose interference with the gantry 10 is prevented, and a current horizontal position of the cradle 20c if the cradle 20 will interfere with the gantry 10. If the interference is predicted, the signal Mu or Md is driven low in order to suspend the variation of the table height. Moreover, a tilt angle variation suspending signal is transmitted to the gantry 10.

When the cradle is controlled as mentioned above, the interference of the cradle 20c with the gantry 10 can be prevented.

For scout scanning, axial cluster scanning, or helical scanning, the control equipment 30 treats the movement of the table 20i as part of the movement of the cradle 20c, and moves the table 20i and cradle 20c concurrently horizontally.

When control is extended as mentioned above, a scanning permissible range can be expanded.

### SECOND EMBODIMENT

The table 201 may be of a telescopic type having two or more table portions nested.

In this case, the aforesaid control sequence adapted to the table 20i is adapted to a table portion that is projected or put into the innermost part of the bore of the gantry.

Many widely different embodiments of the invention may be configured without departing from the spirit and the scope of the present invention. It should be understood that the present invention is not limited to the specific embodiments described in the specification, except as defined in the appended claims.

1. Transportation apparatus comprising:
a cradle which is horizontally moved with a subject lying down thereon;
a table which supports the cradle so that the cradle can be horizontally moved;
a support which bears the table so that the table can be horizontally moved and whose height can be varied;
a horizontal cradle movement device for horizontally moving the cradle;
a horizontal table movement device for horizontally moving the table; and
a control device for controlling and interlocking the horizontal cradle movement device and horizontal table movement device so that the horizontal movements of the cradle and table can be performed concurrently.

2. The transportation apparatus according to claim 1, wherein: the control device determines a target cradle position and a target table position; the horizontal table movement device horizontally moves the table until the table reaches the target table position; the horizontal cradle movement device horizontally moves the cradle until the cradle reaches the target cradle position; and the velocities of the horizontal movements are controlled so that the arrivals of the table and cradle will coincide with each other.

3. The transportation apparatus according to claim 1, wherein when decelerating the table, the control device accelerates the cradle so as to compensate for the deceleration.

4. The transportation apparatus according to claim 1, wherein when horizontally moving the table, the control device concurrently horizontally moves the cradle in an opposite direction by the magnitude of the horizontal movement made by the table.

5. A tomography system comprising:
a gantry that produces tomographic images of a subject;
a cradle which carries the subject, who lies down thereon, into the bore of the gantry;
a table which supports the cradle so that the cradle can be horizontally moved;
a support which bears the table so that the table can be horizontally moved and whose height can be varied;
a horizontal cradle movement device for horizontally moving the cradle;
a horizontal table movement device for horizontally moving the table;
a table height variation device for varying the height of the table; and
a control device for controlling and interlocking the horizontal cradle movement device and horizontal table movement device so that the horizontal movements of the cradle and table can be concurrently performed.

6. The tomography system according to claim 5, wherein: the control device determines a target cradle position and a target table position; the horizontal table movement device horizontally moves the table until the table reaches the target table position; the horizontal cradle movement device horizontally moves the cradle until the cradle reaches the target cradle position; and the velocities of the horizontal movements are controlled so that the arrivals of the table and cradle will coincide with each other.

7. The tomography system according to claim 5, wherein when decelerating the table, the control device accelerates the cradle so as to compensate for the deceleration.

8. The tomography system according to claim 5, wherein when horizontally moving the table, the control device concurrently horizontally moves the cradle in an opposite direction by the magnitude of the horizontal movement made by the table.

9. A tomography system comprising:
a gantry that produces tomographic images of a subject;
a cradle which carries the subject, who lies down thereon, into the bore of the gantry;
a table which supports the cradle so that the cradle can be horizontally moved;
a support which bears the table so that the table can be horizontally moved and whose height can be varied;
a horizontal cradle movement device for horizontally moving the cradle;
a horizontal table movement device for horizontally moving the table;
a table height variation device for varying the height of the table; and
a control device for controlling the horizontal cradle movement device and horizontal table movement device, wherein:
the control device calculates a position, at which the table will not interfere with the gantry but will lie closest to the gantry, on the basis of a current tilt angle and a current table height, and regards the position as a scan-time position at which the table should be located during scanning; and
when a tilt angle or a table height is varied, if interference of the table with the gantry is predicted, the control device pulls the table out of the bore of the gantry.

10. A tomography system comprising:
a gantry that produces tomographic images of a subject;
a cradle which carries the subject, who lies down thereon, into the bore of the gantry;
a table which supports the cradle so that the cradle can be horizontally moved;
a support which bears the table so that the table can be horizontally moved and whose height can be varied;
a horizontal cradle movement device for horizontally moving the cradle;
a horizontal table movement device for horizontally moving the table;
a table height variation device for varying the height of the table; and
a control device for controlling the horizontal cradle movement device and horizontal table movement device, wherein:
the control device determines a scan-time position, at which the table should be located during scanning, on the basis of a region to be scanned or conditions for scanning; and
when a tilt angle or a table height is varied, if the scan-time position is predicted to become improper, the control device pulls the table out of the bore of the gantry.

11. A tomography system comprising:
a gantry that produces tomographic images of a subject;
a cradle which carries the subject, who lies down thereon, into the bore of the gantry;
a table which supports the cradle so that the cradle can be horizontally moved;
a support which bears the table so that the table can be horizontally moved and whose height can be varied;
a horizontal cradle movement device for horizontally moving the cradle;
a horizontal table movement device for horizontally moving the table;
a table height variation device for varying the height of the table; and
a control device for controlling the horizontal cradle movement device and horizontal table movement device, wherein:
the control device calculates a position, at which the table will not interfere with the gantry but will lie closest to the gantry, on the basis of a maximum tilt angle and a current table height, and regards the position as a scan-time position at which the table should be located during scanning; and
when a table height is varied, if interference of the table with the gantry is predicted, the control device pulls the table out of the bore of the gantry.

12. A tomography system comprising:
a gantry that produces tomographic images of a subject;
a cradle which carries the subject, who lies down thereon, into the bore of the gantry;
a table which supports the cradle so that the cradle can be horizontally moved;
a support which bears the table so that the table can be horizontally moved and whose height can be varied;
a horizontal cradle movement device for horizontally moving the cradle;
a horizontal table movement device for horizontally moving the table;
a table height variation device for varying the height of the table; and
a control device for controlling the horizontal cradle movement device and horizontal table movement device, wherein:
the control device calculates a position, at which the table will not interfere with the gantry but will lie closest to the gantry, on the basis of a current tilt angle and a minimum table height permitting the cradle to be put into the bore of the gantry at present, and regards the position as a scan-time position at which the table should be located during scanning; and
when a tilt angle is varied, if interference of the table with the gantry is predicted, the control device pulls the table out of the bore of the gantry.

13. A tomography system comprising:
a gantry that produces tomographic images of a subject;
a cradle which carries the subject, who lies down thereon, into the bore of the gantry;
a table which supports the cradle so that the cradle can be horizontally moved;
a support which bears the table so that the table can be horizontally moved and whose height can be varied;
a horizontal cradle movement device for horizontally moving the cradle;
a horizontal table movement device for horizontally moving the table;
a table height variation device for varying the height of the table; and
a control device for controlling the horizontal cradle movement device and horizontal table movement device, wherein:
the control device calculates a position, at which the table will not interfere with the gantry but will lie closest to the gantry, on the basis of a maximum tilt angle and a minimum table height permitting the cradle to be put into the bore of the gantry with the gantry held at the maximum tilt angle, and regards the position as a scan-time position at which the table should be located during scanning.

14. The tomography system according to claim 9, wherein when a tilt angle or a table height is varied, if interference of the cradle with the gantry is predicted, the control device suspends the variation of the tilt angle or table height.

15. The tomography system according to claim 10, wherein when a tilt angle or a table height is varied, if interference of the cradle with the gantry is predicted, the control device suspends the variation of the tilt angle or table height.

16. The tomography system according to claim 11, wherein when a tilt angle or a table height is varied, if interference of the cradle with the gantry is predicted, the control device suspends the variation of the tilt angle or table height.

17. The tomography system according to claim 12, wherein when a tilt angle or a table height is varied, if interference of the cradle with the gantry is predicted, the control device suspends the variation of the tilt angle or table height.

18. The tomography system according to claim 13, wherein when a tilt angle or a table height is varied, if interference of the cradle with the gantry is predicted, the control device suspends the variation of the tilt angle or table height.
